(19) United States
(12) Patent Application Publication
(10) Pub. No.: US 2025/0332383 A1
(43) Pub. Date: Oct. 30, 2025

(54) CATHETER WITH MARKER DEVICE AND POLYMERIC TIP

(71) Applicants: STRYKER CORPORATION, Portage, MI (US); STRYKER EUROPEAN OPERATIONS LIMITED, Carrigtwohill (IE)

(72) Inventors: Enkhsanaa Batsukh, Portage, MI (US); Huey Chan, San Jose, CA (US); John Lackey, Portage, MI (US); Muriel Boling, Portage, MI (US); Madalyn Kern, Portage, MI (US); Lee Puckett, Portage, MI (US)

(73) Assignees: STRYKER CORPORATION, Portage, MI (US); STRYKER EUROPEAN OPERATIONS LIMITED, Carrigtwohill (IE)

(21) Appl. No.: 19/256,095

(22) Filed: Jun. 30, 2025

Related U.S. Application Data
(63) Continuation of application No. PCT/US2024/045889, filed on Sep. 9, 2024.
(60) Provisional application No. 63/583,519, filed on Sep. 18, 2023.

(51) Int. Cl. A61M 25/01; A61M 25/00

(52) U.S. Cl. CPC A61M 25/0108; A61M 25/0054

(57) ABSTRACT

A marker device includes: a ring structure having a distal end, a proximal end, and a body, wherein the ring structure is made from a radiopaque material; wherein the distal end comprises protruding elements disposed circumferentially around an axis of the ring structure; and wherein the proximal end is configured to couple with, or extends from, a tubular structure. A catheter includes: a marker device having a ring structure with a distal end, a proximal end, and a body, wherein the ring structure is made from a radiopaque material; and a polymeric tube distal to the marker device, wherein the polymeric tube comprises a distal tip extending circumferentially around a longitudinal axis of the catheter; wherein the distal end comprises a distal ring tip extending circumferentially around the longitudinal axis, and wherein the distal ring tip does not completely lie in any plane that is perpendicular to the longitudinal axis.

### RELATED APPLICATION DATA

This application is a continuation of International Patent Application No. PCT/US2024/045889, filed on Sep. 9, 2024, which claims priority to, and the benefit of, U.S. Provisional Patent Application No. 63/583,519 filed on Sep. 18, 2023.

### FIELD

The present disclosure relates generally to minimally invasive medical devices, and more specifically to catheters.

### BACKGROUND

The use of intravascular catheters for accessing and treating various types of diseases, such as vascular defects, is well-known. For example, a suitable intravascular catheter may be inserted into the vascular system of a patient. Commonly used vascular application to access a target site in a patient involves inserting a guidewire through an incision in the femoral artery near the groin, and advancing the guidewire until it reaches the target site. Then, a catheter is advanced over the guidewire until an open distal end of the catheter is disposed at the target site. Simultaneously or after placement of the distal end of the catheter at the target site, an intravascular implant is advanced through the catheter via a delivery wire.

For trackability, the flexibility of the distal segment of the catheter is critical. Currently, catheters have a solid markerband made from radiopaque material at the distal end of the catheter, which allows the catheter to be imaged. The solid markerband is a solid ring that joins to a distal end of a hypotube. The solid ring stiffens the catheter, making it difficult to track and to deflect away from branching arteries such as the ophthalmic artery. Thus, new marker device that reduces the stiffness of the catheter would be desirable.

Also, in certain applications, such as neurovascular treatment, the catheters are required to navigate tortuous and intricate vasculature. By using an appropriately sized device having the requisite performance characteristics, such as “pushability” “steerability”, “torqueability” and most important, distal tip flexibility, virtually any target site in the vascular system may be accessed, including that within the tortuous cerebral and peripheral vasculature. Further, the forces applied at the proximal end of these catheters should be transferred to the distal ends for suitable pushability (axial rigidity) and torqueability (rotation). Achieving a balance between these features is highly desirable, but difficult.

In addition, a catheter may have a lumen with a certain cross-sectional shape. During use, the catheter may be bent. For example, tensioning wire may be operated to bend the catheter, and/or the catheter may be bent via guidewire or by a curvature of an anatomy. The bending of the catheter causes a compression on one side of the catheter and tension on an opposite side of the catheter. In some cases, due to the compression associated with the bending of the catheter, the catheter may kink, thereby collapsing the lumen of the catheter. Designing a catheter to resist such kinking while achieving certain bending flexibility and torsional rigidity is very difficult to accomplish.

### SUMMARY

A marker device includes: a ring structure having a distal end, a proximal end, and a body extending between the distal end and the proximal end, wherein the ring structure is made from a radiopaque material; wherein the distal end of the ring structure comprises protruding elements disposed circumferentially around an axis of the ring structure; and wherein the proximal end of the ring structure is configured to couple with, or extends from, a tubular structure.

Optionally, the proximal end of the ring structure comprises tabs disposed circumferentially around the axis of the ring structure.

Optionally, the tabs are configured to be welded to the tubular structure.

Optionally, the protruding elements comprise respective curvilinear tip surfaces.

Optionally, the distal end of the ring structure further comprises curvilinear trough surfaces, wherein each of the curvilinear trough surfaces is disposed between two adjacent ones of the curvilinear tip surfaces.

Optionally, the curvilinear tip surfaces and the curvilinear trough surfaces together form a sinusoidal profile extending circumferentially around the axis of the ring structure.

Optionally, the protruding elements comprise respective elongated elements.

Optionally, the protruding elements at the distal end of the ring structure extend proximally to the body of the ring structure.

Optionally, each of the protruding elements has a longitudinal length measured in a direction parallel to the axis of the ring structure, wherein a ratio calculated by dividing the longitudinal length by a total longitudinal length of the ring structure is at least 0.5.

Optionally, the protruding elements comprise four or more protruding elements.

Optionally, the distal end of the ring structure has a laser-cut edge.

Optionally, the ring structure has a closed-loop configuration.

Optionally, the ring structure has an open-loop configuration.

Optionally, the ring structure has a plurality of holes disposed circumferentially at the body of the ring structure.

Optionally, at least one of the holes has an elongated configuration extending at least partially around the axis of the ring structure.

A catheter includes the marker device, and a first polymer layer disposed over an exterior surface of the ring structure, wherein parts of the first polymer layer extend into the holes of the ring structure.

Optionally, the first polymer layer extends distally past a distal tip of the marker device to form a polymeric tip.

Optionally, the catheter further includes a second polymer layer disposed at an interior surface of the ring structure.

Optionally, the holes are distributed longitudinally with respect to the body of the ring structure.

A catheter includes the marker device, and the tubular structure, wherein the tubular structure is configured to provide axial stiffness, bending stiffness, torsional stiffness, or any combination of the foregoing, for the catheter.

Optionally, the tubular structure comprises a hypotube.

Optionally, the catheter further includes a first polymer layer disposed over an exterior surface of the ring structure, and over at least a part of an exterior surface of the tubular structure.

Optionally, the first polymer layer extends distally past a distal tip of the marker device to form a polymeric tip.

Optionally, the polymeric tip comprises a polymeric tube that is distal to the marker device.

Optionally, the polymeric tube comprises a distal tip extending circumferentially around a longitudinal axis of the catheter.

Optionally, the distal tip of the polymeric tube has a profile that corresponds geometrically with a profile at a tip of the marker device.

Optionally, the distal tip of the polymeric tube has a profile that does not correspond geometrically with a profile at a tip of the marker device.

Optionally, the distal tip of the polymeric tube completely lies within a plane.

Optionally, the plane is perpendicular to a longitudinal axis of the catheter.

Optionally, the polymeric tube comprises a plurality of flanges disposed circumferentially around a longitudinal axis of the catheter.

Optionally, the flanges of the polymeric tube are configured to move radially away from the longitudinal axis of the catheter.

Optionally, distances from the distal tip of the polymeric tube to a tip of the marker device measured at different circumferential positions at the polymeric tube are the same.

Optionally, distances from the distal tip of the polymeric tube to a tip of the marker device measured at different circumferential positions at the polymeric tube are different.

A catheter includes: a marker device having a ring structure, the ring structure having a distal end, a proximal end, and a body extending between the distal end and the proximal end, wherein the ring structure is made from a radiopaque material; and a polymeric tube distal to the marker device, wherein the polymeric tube comprises a distal tip extending circumferentially around a longitudinal axis of the catheter; wherein the distal end of the ring structure comprises a distal ring tip extending circumferentially around the longitudinal axis of the catheter, and wherein the distal ring tip does not completely lie in any plane that is perpendicular to the longitudinal axis of the catheter.

Optionally, the distal tip of the polymeric tube has a profile that corresponds geometrically with a profile of the distal ring tip.

Optionally, the distal tip of the polymeric tube has a profile that does not correspond geometrically with a profile of the distal ring tip.

Optionally, the distal tip of the polymeric tube completely lies within a plane. Optionally, the plane is perpendicular to the longitudinal axis of the catheter.

Optionally, the polymeric tube comprises a plurality of flanges disposed circumferentially around the longitudinal axis of the catheter.

Optionally, the flanges of the polymeric tube are configured to move radially away from the longitudinal axis of the catheter.

Optionally, distances from the distal tip of the polymeric tube to the distal ring tip measured at different circumferential positions at the polymeric tube are the same.

Optionally, distances from the distal tip of the polymeric tube to the distal ring tip measured at different circumferential positions at the polymeric tube are different.

Optionally, the distal end of the ring structure comprises protruding elements disposed circumferentially around the longitudinal axis of the catheter.

Optionally, the proximal end of the ring structure comprises tabs disposed circumferentially around the longitudinal axis of the catheter.

Optionally, the tabs are configured to be welded to a tubular structure.

Optionally, the protruding elements comprise respective curvilinear tip surfaces that are part of the distal ring tip.

Optionally, the ring structure further comprises curvilinear trough surfaces, wherein each of the curvilinear trough surfaces is disposed between two adjacent ones of the curvilinear tip surfaces.

Optionally, the curvilinear tip surfaces and the curvilinear trough surfaces together form a sinusoidal profile extending circumferentially around the longitudinal axis of the catheter.

Optionally, the protruding elements comprise respective elongated elements.

Optionally, the protruding elements at the distal end of the ring structure extend proximally to the body of the ring structure.

Optionally, each of the protruding elements has a longitudinal length measured in a direction parallel to the longitudinal axis of the catheter, wherein a ratio calculated by dividing the longitudinal length by a total longitudinal length of the ring structure is at least 0.5.

Optionally, the protruding elements comprise four or more protruding elements.

Optionally, the distal end of the ring structure has a laser-cut edge.

Optionally, the ring structure has a closed-loop configuration.

Optionally, the ring structure has an open-loop configuration.

Optionally, the ring structure has a plurality of holes disposed circumferentially at the body of the ring structure.

Optionally, at least one of the holes has an elongated configuration extending at least partially around the longitudinal axis of the catheter.

Optionally, the catheter further includes a first polymeric layer disposed circumferentially around an exterior surface of the ring structure.

Optionally, the polymeric tube is integral with the first polymeric layer.

Optionally, the catheter further includes a second polymeric layer disposed at an inner surface of the ring structure.

Optionally, the polymeric tube is also integral with the second polymeric layer.

Optionally, the catheter further includes a polymeric layer disposed at an inner surface of the ring structure.

Optionally, the polymeric tube is integral with the polymeric layer.

A catheter includes: a tubular structure having a distal end, a proximal end, and a body extending between the distal end and the proximal end, wherein the tubular structure is made from a metal or an alloy; and a polymeric tube distal to the tubular structure, wherein the polymeric tube comprises a plurality of protrusions disposed circumferentially around a longitudinal axis of the catheter.

Optionally, the protrusions comprise respective flaps.

Optionally, the protrusions comprise respective elongated members.

Optionally, the protrusions are configured to move radially away from the longitudinal axis of the catheter.

Optionally, the polymeric tube comprises a proximal tube end that abuts the distal end of the tubular structure.

Optionally, the catheter further includes a marker disposed between the tubular structure and the polymeric tube.

Optionally, the catheter further includes a first polymeric layer disposed circumferentially around an exterior surface of the tubular structure.

Optionally, the polymeric tube is integral with the first polymeric layer.

Optionally, the catheter further includes a second polymeric layer disposed at an inner surface of the tubular structure.

Optionally, the polymeric tube is also integral with the second polymeric layer.

Optionally, the catheter further includes a polymeric layer disposed at an inner surface of the tubular structure.

Optionally, the polymeric tube is integral with the polymeric layer.

Optionally, the tubular structure comprises a hypotube.

In one or more embodiments described herein, the catheter includes a first polymer layer disposed around a marker device and/or a tubular structure.

Optionally, the first polymer layer comprises a first segment and a second segment proximal the first segment.

Optionally, the first segment is made from NEUSoft.

Optionally, the NEUSoft comprises NEUSoft 62A.

Optionally, the second segment is made from Pebax.

Optionally, the Pebax comprises Pebax 25D, Pebax 35D, or Pebax 45D.

Optionally, the first polymer layer further comprises a third segment.

Optionally, the second segment is made from a first Pebax, and the third segment is made from a second Pebax that is different from the first Pebax.

Optionally, the first polymer layer further comprises a fourth segment.

Optionally, the second segment is made from a first Pebax, the third segment is made from a second Pebax that is different from the first Pebax and the fourth segment is made from a third Pebax that is different from the first Pebax and that is different from the second Pebax.

Optionally, the catheter further includes a second polymer layer disposed at an interior surface of the ring structure, and over at least a part of an interior surface of the hypotube.

Optionally, the second polymer layer comprises a first segment and a second segment proximal the first segment.

Optionally, the first segment of the second polymer layer is made from NEUSoft.

Optionally, the NEUSoft comprises NEUSoft 62A.

Optionally, the second segment of the second polymer layer is made from PTFE.

Optionally, a first segment of the first polymer layer and a first segment of the second polymer layer are made from a same material.

Optionally, the first segment of the first polymer layer and the first segment of the second polymer layer are closer to a distal end of the catheter than to a proximal end of the catheter.

Optionally, the same material comprises NEUSoft.

Optionally, a second segment of the first polymer layer and a second segment of the second polymer layer are made from different respective materials, wherein the second segment of the first polymer layer is proximal to the first segment of the first polymer layer, and wherein the second segment of the second polymer layer is proximal to the first segment of the second polymer layer.

In one or more embodiments described herein, the tubular structure comprises a first ring element, a second ring element, a third ring element, a first set of connecting members disposed between the first ring element and the second ring element, and a second set of connecting members disposed between the second ring element and the third ring element.

Optionally, one of the connecting members in the first set and one of the connecting members in the second set form a junction with the second ring element.

In one or more embodiments described herein, the junction has a cross-configuration, wherein a part of the cross-configuration is formed by a portion of the second ring element.

Optionally, the first set of connecting members comprises no more than four connecting members.

Optionally, one of the connecting members in the first set has a width and a thickness, wherein the width is less than the thickness, wherein the thickness is measured in a radial direction with respect to the tubular structure, and wherein the width is measured in a direction that is perpendicular to the radial direction.

Other and further aspects and features of embodiments will become apparent from the ensuing detailed description in view of the accompanying figures.

### BRIEF DESCRIPTION OF THE FIGURES

FIG. 1 illustrates a catheter in accordance with some embodiments.

FIG. 2 illustrates a distal segment of the catheter of FIG. 1, particularly showing the distal segment of the catheter having a marker device and a tubular structure.

FIG. 3 illustrates the marker device of FIG. 2.

FIGS. 4-5 illustrate a variation of the marker device of FIG. 3, particularly showing the marker device having multiple tabs.

FIG. 6 illustrates a variation of the marker device of FIG. 4, particularly showing the marker device having multiple openings.

FIGS. 7-8 illustrate a variation of the distal segment of the catheter of FIG. 1, particularly showing the distal segment of the catheter having a marker device with multiple holes disposed circumferentially around an axis of the marker device.

FIGS. 9-11 illustrate a variation of the marker device of FIG. 2, particularly showing the marker device having elongated protrusions disposed circumferentially around an axis of the marker device.

FIG. 12 illustrates another example of the distal segment of the catheter of FIG. 2.

FIG. 13 illustrates an example of a polymeric tip at the distal segment of FIG. 2, particularly showing the polymeric tip having variable lengths around a circumference of the distal segment.

FIG. 14 illustrates a variation of the polymeric tip of FIG. 13.

FIGS. 15A-15B illustrate another variation of the polymeric tip of FIG. 13.

FIG. 16 illustrates another variation of the polymeric tip of FIG. 13.

FIG. 17 illustrates a distal segment of the catheter of FIG. 1.

FIGS. 18A-18B illustrate a part of a tubular structure of the catheter of FIG. 1.

FIGS. 19A-19B illustrate a bending of the tubular structure of FIG. 18A.

FIGS. 20A-20B illustrate a bending of another tubular structure that is different from the tubular structure of FIG. 18A.

FIG. 21 illustrates another tubular structure.

FIG. 22 illustrates a bending of the tubular structure of FIG. 21.

FIG. 23 illustrates a tensioning of the tubular structure of FIG. 21.

FIG. 24 illustrates a tube segment having the tubular structure of FIG. 21, particularly showing the tube segment being bent.

FIG. 25 illustrates the tube segment of FIG. 24, particularly showing the tube segment being tensioned.

FIG. 26 illustrates the tube segment of FIG. 24, particularly showing the tube segment being tested for kink resistance.

### DETAILED DESCRIPTION

Various embodiments are described hereinafter with reference to the figures. It should be noted that the figures may or may not be drawn to scale, and that elements of similar structures or functions are represented by the same reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. In some cases, the term “about” may refer to a range of values that are within +/−10% of a value. For example, a value of 2 or a value of about 2 may refer to any value that is within the range of 2+/−10% (=2+/−0.2=1.8 to 2.2).

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

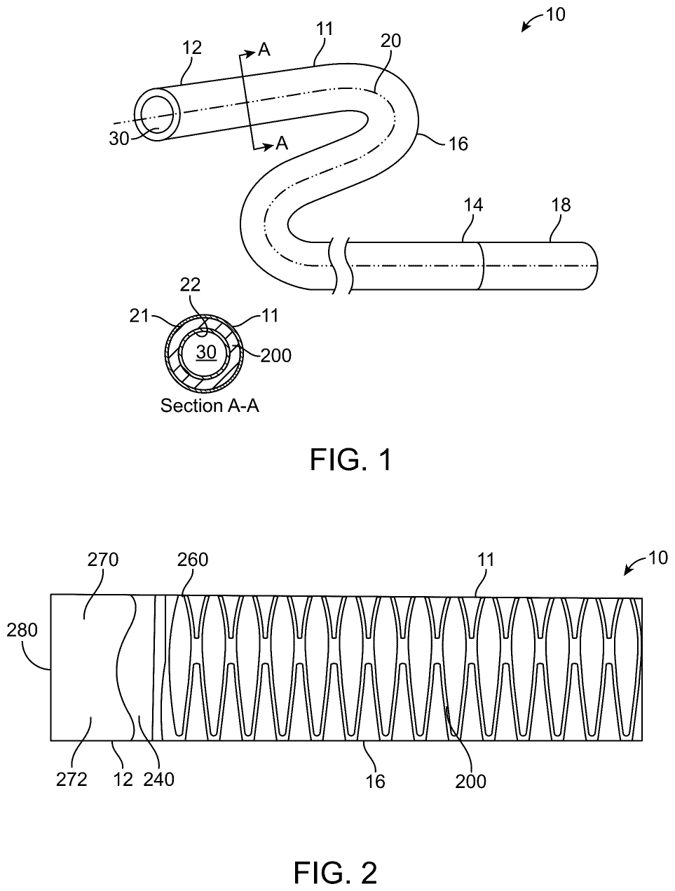

FIG. 1 illustrates a catheter 10 in accordance with some embodiments. The catheter 10 includes a tube 11 having a distal end 12, a proximal end 14, and a tube body 16 extending between the distal end 12 and the proximal end 14. The catheter also includes a handle 18 attached to the proximal end 14 of the tube 11.

The tube 11 includes an outer surface 21, and inner surface 22, and a lumen defined by the inner surface. The tube 11 also includes a tubular structure 200 configured to provide certain stiffness for the tube 11. As shown in the figure, the tubular structure 200 is disposed between the outer surface 21 and the inner surface 22 of the tube 11, so that the tubular structure 200 is embedded within a wall of the tube 11. In other embodiments, the tubular structure 200 may be on the outer surface 21, or on the inner surface 22 of the tube 11. The tubular structure 200 has a distal end, a proximal end, and a body extending between the distal end and the proximal end. The tubular structure 200/catheter 10 also has a longitudinal axis 20 defined by the distal end and the proximal end of the tubular structure 200.

In the illustrated embodiments, the lumen 30 of the catheter 10 has a cross-sectional shape when the catheter 10 is in a relaxed state. The tubular structure 200 is configured to maintain the cross-sectional shape of the lumen 30 during bending of the catheter 10, so that the catheter 10 will not kink. Optionally, the tubular structure 200 may also be configured to provide axial stiffness and/or torsional stiffness for the catheter 10.

FIG. 2 illustrates a distal segment of the catheter 10 of FIG. 1, particularly showing the distal segment of the catheter 10 having a marker device 240 and a tubular structure 200. The tubular structure 200 is configured to provide axial stiffness, bending stiffness, torsional stiffness, or any combination of the foregoing, for the catheter 10. In some cases, the tubular structure 200 may comprise a hypotube. The distal segment of the catheter 10 also includes a polymer layer 260 disposed circumferentially around the marker device 240 and/or the tubular structure 200. The distal segment of the catheter 10 further includes a polymeric tip 270. The polymeric tip 270 comprises a polymeric tube 272 having a distal tip 280.

### Marker Device

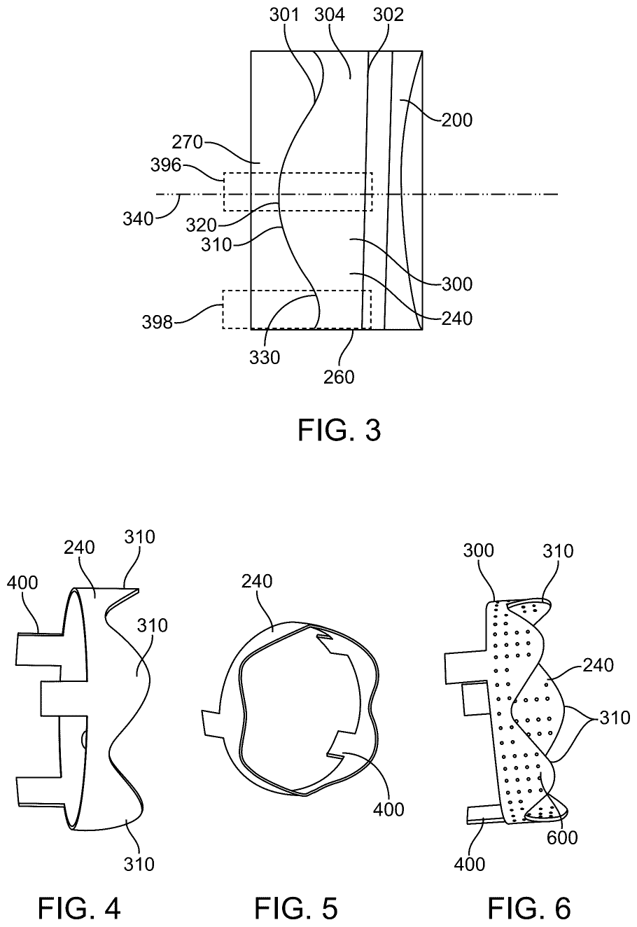

FIG. 3 illustrates the marker device 300 of FIG. 2. As shown in the figure, the marker device 240 includes: a ring structure 300 having a distal end 301, a proximal end 302, and a body 304 extending between the distal end 301 and the proximal end 302. The ring structure 300 may be made from a radiopaque material, or any material that may allow visualization of the marker device 300 using any imaging technique (e.g., x-ray, CT, MRI, ultrasound, camera, etc.). The distal end 301 of the ring structure 300 comprises protruding elements 310 disposed circumferentially around an axis 340 of the ring structure 300. The proximal end 302 of the ring structure 300 is configured to couple with, or extends from, the tubular structure 200.

As shown in FIG. 3, the protruding elements 310 comprise respective curvilinear tip surfaces 320. The distal end 301 of the ring structure 300 further includes curvilinear trough surfaces 330. As shown in the figure, each of the curvilinear trough surfaces 330 is disposed between two adjacent ones of the curvilinear tip surfaces 320. In some cases, the curvilinear tip surfaces 320 and the curvilinear trough surfaces 330 together form a sinusoidal profile extending circumferentially around the axis 340 of the ring structure 300. In some cases, the number of the tip surfaces 320/protruding elements 310 may be two, three, four, five, six, seven, or eight. In other cases, the number of tip surfaces 320/protruding elements 310 may be more than eight.

In other embodiments, each protruding elements 310 may have a shape that is different from the example shown. For example, in other embodiments, each protruding element 310 may have a rectangular shape, a partial circular shape, an elongated shape, etc. Also, in the illustrated embodiments, each protruding element 310 has a length and a width, wherein the length is measured parallel to the longitudinal axis 340 and is shorter than the width of the protruding element 310. In other embodiments, each protruding element 310 may have a length and a width, wherein the length is measured parallel to the longitudinal axis 340 and is longer than the width of the protruding element 310.

As illustrated in the above examples, the protruding elements 310 have respective distal tips (having respective edges or tip surfaces 320) facing distally. In some embodiments, the distal end of the ring structure 300 may have a laser-cut edge.

The marker device 240 is advantageous because it is more flexible compared to another marker device having the same longitudinal length and thickness as those of the marker device 240, and being made from the same material as that of the marker device 240. By not having some materials in an alternating pattern circumferentially around the marker device 240, the marker device 240 has a “flower petal” shape having multiple protruding elements 310 (e.g., “petals”). Such configuration of the marker device 240, together with the softer polymeric tip 270, results in alternating sections 396, 398 at the distal end of the catheter 10, wherein the section 296 has more stiff marker material (from the marker device 240) for allowing the catheter 10 to resist ovalization, and the section 398 has more polymeric material (from the polymeric tip 270) for allowing the catheter 10 to achieve trackability.

The marker device 240 is also advantageous because it allows visualization under imaging, such as fluoroscopy, x-ray, CT, etc. The circumferentially alternating pattern achieved due to the protruding elements 310 allows visualization of torsional movement of the catheter. Also, in one implementation, the polymeric tip 270 extends less than 3 mm, or more preferably less than 2 mm, and even more preferably less than 1 mm, beyond the distal-most tip of the marker device 240. Such configuration may prevent, or at least reduce the risk of, collapsing of the polymeric tip 270 during aspiration and/or tracking. In addition, by having this “flush cut” design, a physician is able to know exactly where the catheter tip is during a medical procedure.

In some cases, the proximal end 302 of the ring structure 300 may optionally include tabs 400 disposed circumferentially around the axis 340 of the ring structure 300 (FIGS. 4-5). The tabs 400 are configured to be secured to the tubular structure 200, e.g., via weld (such as spot weld), adhesive, mechanical coupling, etc. The tabs 400 are advantageous because they provide a spacing between the marker device 240 and the tubular structure 200, which spacing allows for bending of the catheter 10. In the illustrated example, the marker device 240 has three tabs. In other cases, the marker device 240 may have two tabs, or more than three tabs.

In some cases, the ring structure 300 of the marker device 240 has a closed-loop configuration. In such cases, the ring structure 300 has a continuous wall extending circumferentially around the axis 340. In other cases, the ring structure 300 of the marker device 240 has an open-loop configuration. For example, the ring structure 300 may have a slit extending longitudinally from the distal end 301 to the proximal end 302. Such configuration may provide additional flexibility for the catheter 10.

FIG. 6 illustrates a variation of the marker device 240 of FIG. 4, particularly showing the marker device having multiple openings 600. The openings 600 may be holes disposed circumferentially at the body 304 of the ring structure 300. The holes may be tiny through holes extending through a wall thickness of the ring structure 300. Each hole may be a circular hole, an elliptical hole, a square hole, a rectangular hole, or any hole with any geometric shape or any user-define shape. As shown in the figure, the openings 600 (holes) are distributed in both the longitudinal direction (i.e., the direction that is parallel to the longitudinal axis 340), and the circumferential direction (i.e., the direction extending circumferentially and being perpendicular to the longitudinal axis 340). In other cases, the marker device 240 may have only a single row of the openings 600 extending circumferentially around the axis 340. In such cases, the openings 600 are distributed circumferentially, and are not distributed longitudinally.

In one implementation, the openings 600 may be tiny holes having a cross-sectional dimension that is 1 mm or smaller. In other cases, each of the openings 600 may have a cross-sectional dimension that is larger than 1 mm. The openings 600 may be made using laser, punching, drilling, molding, etc. The openings 600 are advantageous because they allow material from the first polymer layer 260 to anchor against the marker device 240. In some cases, the catheter 10 may include a second polymer layer (e.g., the polymer layer 262 of FIG. 17). In such cases, the openings 600 allow the first polymer layer 260 to bond to the second polymer layer 262, thereby preventing or reducing the risk of delamination (e.g., separation between the first and second polymer layers).

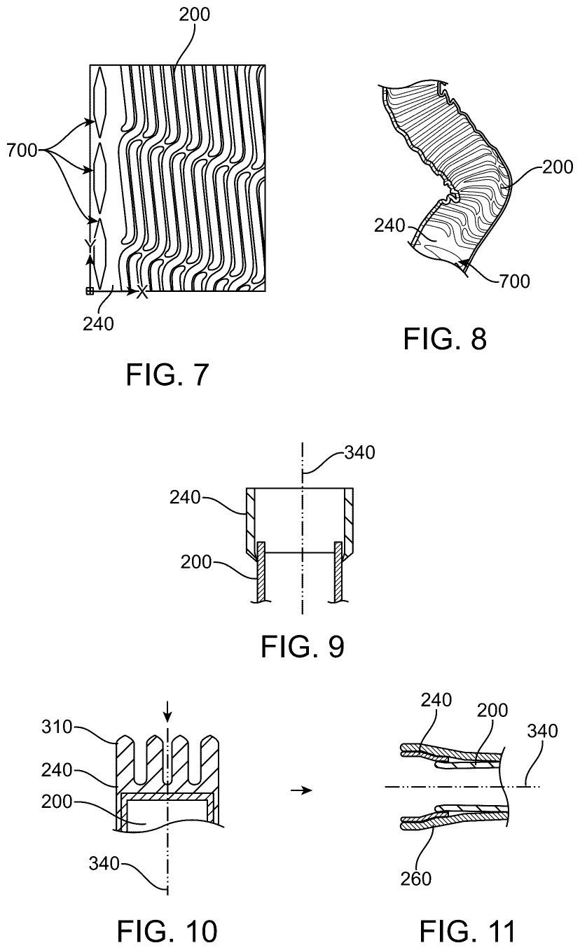

FIGS. 7-8 illustrate a variation of the distal segment of the catheter 10 of FIG. 1, particularly showing the distal segment of the catheter 10 having a marker device 240 with multiple openings 700 disposed circumferentially around an axis of the marker device 240/catheter 10 (e.g., the longitudinal axis 340). Each opening 700 is a hole having an elongated configuration extending at least partially around the axis 340 of the ring structure 300. The openings 700 are advantageous because they may provide a distinct geometric feature for allowing the marker device 240 to be identified via imaging. The openings 700 are also advantageous because they may provide additional flexibility for the catheter 10. For example, the marker device 240 with the openings 700 will allow the catheter 10 to be more flexible (e.g., in bending) compared to another marker device having the same geometry (e.g., size and shape) but without the openings 700. The openings 700 at the marker device 240 are also advantageous because they allow the marker device 240 to bend and/or buckle with less force, they may reduce track force, and may improve trackability for the catheter 10. The number, size, shape, and positions of the openings 700 may be configured to achieve one or more mechanical performance for the catheter 10.

In one or more embodiments described herein, the catheter 10 may include a first polymer layer (e.g., the polymer layer 260) is disposed over an exterior surface of the ring structure 300 and/or at least a part of the tubular structure 200. In such cases, parts of the first polymer layer 260 may extend into the openings 600/700 (e.g., holes) of the ring structure 300. The first polymer layer 260 may extend partly into the openings 600/700 of the ring structure 300, or may extend into the openings 600/700 completely through the wall thickness of the ring structure 300. The first polymer layer 260 may extend distally past a distal tip of the marker device 240 to form the polymeric tip 270, or to couple with the polymeric tip 270.

Also, in some cases, a second polymer layer 262 (shown in FIG. 17) may be disposed at an interior surface of the ring structure 300. The second polymer layer 262 may extend partly into the openings 600/700 of the ring structure 300. In such cases, material from the second polymer layer 262 may join and connect to the material from the first polymer layer 260 at a location that is inside the opening 600/700. Alternatively, the second polymer layer 262 may extend into the openings 600/700 completely through the wall thickness of the ring structure 300 to join and connect to the first polymer layer 260. In some cases, the second polymer layer 262 may extend distally past a distal tip of the marker device 240 to form the polymeric tip 270, or to couple with the polymeric tip 270.

FIGS. 9-11 illustrate a variation of the marker device 240 of FIG. 2, particularly showing the marker device 240 having elongated protrusions 310 disposed circumferentially around an axis of the marker device 240 (e.g., the longitudinal axis 340). As shown in FIG. 10, the protrusions 310 (protruding elements) comprise respective elongated elements. The protruding elements 310 are at the distal end of the ring structure 300 and extend proximally to the body of the ring structure 300. In some cases, the protruding elements 310 may be considered as a part of the distal end. Also, in some cases, the protruding elements 310 may be considered as a part of the body of the ring structure 300. In one exemplary implementation, each of the protruding elements 310 has a longitudinal length measured in a direction parallel to the axis 340 of the ring structure 300, wherein a ratio calculated by dividing the longitudinal length by a total longitudinal length of the ring structure 300 is at least 0.5.

The elongated protrusions 310 of the marker device 240 is advantageous because they may provide a distinct geometric feature for allowing the marker device 240 to be identified via imaging. The elongated protrusions 310 are also advantageous because they may provide additional flexibility for the catheter 10. For example, the marker device 240 with the elongated protrusions 310 will allow the catheter 10 to be more flexible (e.g., in bending) compared to another marker device having the same geometry (e.g., size and shape) but without the cutouts forming the protrusions 310. Also, the elongated protrusions 310 may make the catheter distal end to be more distensible in order to assist with clot ingestion. In some cases, the length and/or shape of each elongated protrusion 310 may be configured for tip distensibility.

As shown in FIG. 11, the catheter 10 also includes a first layer 260 disposed circumferentially around the marker device 240 (e.g., on the exterior surface of the marker device 240) and the tubular structure 200. In some cases, the catheter 10 may also include a second layer disposed on an inner surface of the marker device 240 and/or the tubular structure 200. Also, in some cases, the catheter 10 may include the polymeric tip 270 that is distal to the marker device 240. In such cases, the first layer 260 and/or the second layer may connect to, or may extend from, the polymeric tip 270. In one implementation, the polymeric tip 270 may be integral with the first layer 260 and/or the second layer. The polymeric tip 270 may be made from the same material as the first layer 260 or the second layer. Alternatively, the polymeric tip 270 may be made from a material that is different from that of the first layer 260 or the second layer.

In some cases, the ring structure 300 may be connected to the tubular structure 200 via a weld, or a connector, or an adhesive, etc. The ring structure 300 may surrounds the tubular structure 200 (like that shown in FIG. 9), and may secure to an exterior surface of the tubular structure 200. Alternatively, the ring structure 300 may be placed partially within the central lumen of the tubular structure 200, and may be secured to an inner surface of the tubular structure 200. In a further alternative technique, the proximal end of the ring structure 300 may face the distal end of the tubular structure 200 to radially align the wall of the ring structure 300 and the wall of the tubular structure 200. In other cases, the ring structure 300 may be formed integrally with the tubular structure 200. For example, the ring structure 300 and the tubular structure 200 may be molded together. Alternatively, the ring structure 300 and the tubular structure 200 may be formed together by obtaining a tube, and laser cutting the tube to form the ring structure 300 and the tubular structure 200.

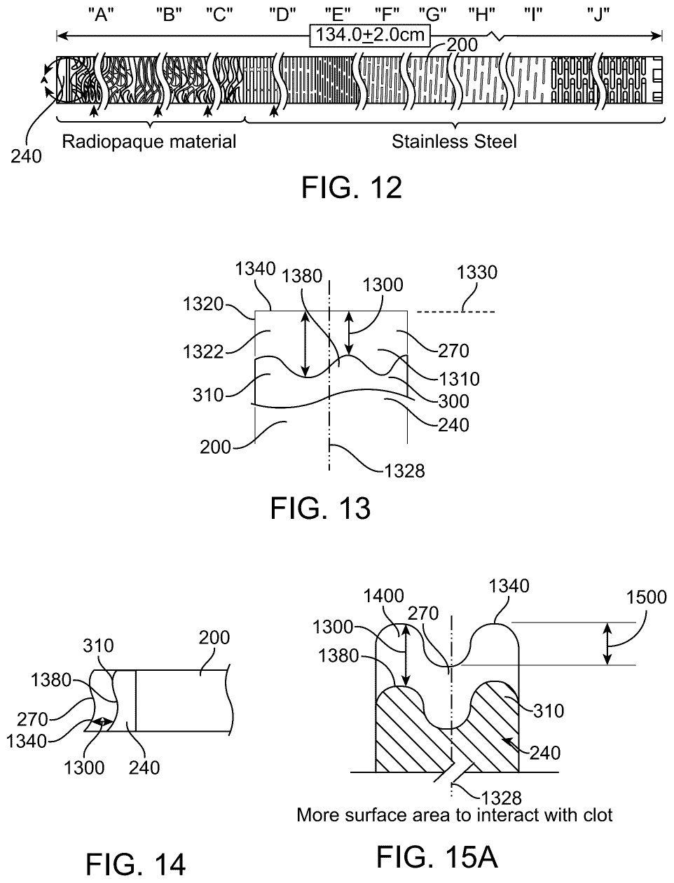

FIG. 12 illustrates another example of the distal segment of the catheter 10 of FIG. 2. As shown in the example, the marker device 240 is coupled to, or extends from, the tubular structure 200.

In the illustrated embodiments, the tubular structure 200 has multiple sections A-J aligned longitudinally. The sections A-J have different respective cut-patterns, which provide different respective stiffnesses (or flexibilities) for the sections A-J. In some cases, each of the sections may be implemented using a tube segment with cutouts, and the tube segments for the sections may be coupled together along the longitudinal axis. In other cases, two or more segments may be implemented with a tube segment. In further cases, all of the segments implementing the entirety of the tubular structure 2000 may be implemented using a single tube segment.

In the illustrated embodiments, part of the tubular structure 200 is made from radiopaque material like that of the marker device 240. In particular, sections A-C of the tubular structure 200 are made from radiopaque material. In other cases, there may be fewer than three sections (e.g., two sections or one section) of the tubular structure 200 that are made from opaque material. In some cases, the marker device 240 may be considered as having the tubular section(s) that is made from opaque materials. The configuration of the design of FIG. 12 is advantageous because it extends the visible length of the catheter under imaging. It is also advantageous because a longer tube (i.e., longer than the length of the traditional markerband) made from the softer radiopaque material may be laser-cut to enable the catheter 10 to have the desired mechanical characteristic(s), such as pushability, bending stiffness, torsional stiffness, trackability, etc., or any combination of the foregoing.

In some embodiments, a first segment of the tubular structure 200 may be more flexible (e.g., having more bending flexibility, axial flexibility, torsional flexibility, or any combination of the foregoing) than a second segment of the tubular structure 200, wherein the second segment is proximal to the first segment. Also, in some cases, the second segment may be more flexible than a third segment of the tubular structure 200, wherein the third segment is proximal to the second segment. In some cases, the bending stiffness of the tubular structure 200 reduces from a proximal end of the tubular structure 200 to a distal end of the tubular structure 200.

It should be noted that the tubular structure 200 is not limited to having all of the segments A-J. In other cases, the tubular structure 200 may have fewer than ten segments (e.g., 1, 2, 3, 4, 5, 6, 7, 8, or 9 segments).

### Polymeric Tip

FIG. 13 illustrates the polymeric tip 270 at the distal segment of FIG. 2, particularly showing the polymeric tip 270 having variable lengths 1300 around a circumference of the distal segment. In the illustrated embodiments, the polymeric tip 270 is in a form of a polymeric tube 1310 that is located distal to the marker device 240 and/or the tubular structure 200. The polymeric tube 1310 (or the polymeric tip 270) has a distal tube end 1320 and a proximal tube end 1322. The distal tube end 1320 of the polymeric tube 1310 (or the polymeric tip 270) has a distal tip 1340 facing distally. The distal tip 1340 may be in a form of an edge or surface.

As shown in the figure, the distal tip 1340 of the polymeric tube 1310 extends circumferentially around a longitudinal axis 1328 of the catheter 10 (or the axis 340 of the marker device 240). At least a part of the polymeric tube 1310 is distal to the marker device 240. The ring structure 300 of the marker device 240 has a distal end with a distal ring tip 1380 extending circumferentially around the longitudinal axis 1328 of the catheter 10. In the illustrated example, the distal ring tip 1380 does not completely lie in any plane that is perpendicular to the longitudinal axis 1328 of the catheter 10. In other cases, the distal ring tip 1380 may completely lie in a plane that is perpendicular to, or that forms an acute angle with, the longitudinal axis 1328 of the catheter 10.

As shown in FIG. 13, the distances 1300 from the distal tip 1340 of the polymeric tube 1310 to the distal ring tip 1380 measured at different circumferential positions at the polymeric tube 1310 are the same. Also, as shown in the figure, the distal tip 1340 of the polymeric tube 1310 completely lies within a plane 1330. The plane 1330 may be perpendicular to the longitudinal axis 1328 of the catheter 10. Alternatively, the plane 1330 may form an acute angle with respect to the longitudinal axis 1328 of the catheter 10.

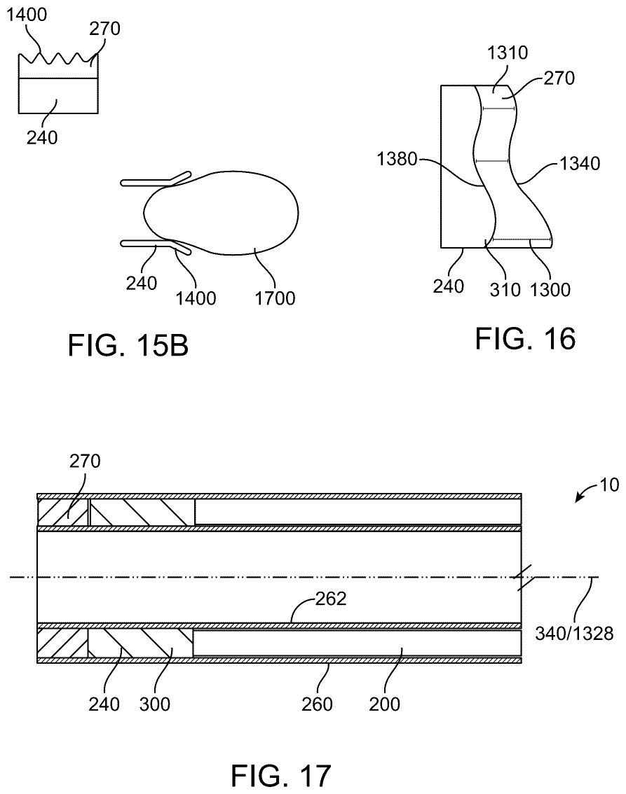

In the illustrated embodiments, the distal tip 1340 of the polymeric tube 1310 has a profile that does not correspond geometrically with a profile of the distal ring tip 1380. In other embodiments, the distal tip 1340 of the polymeric tube 1310 has a profile that corresponds geometrically with a profile of the distal ring tip 1380 (FIGS. 14-15).

As shown in FIG. 14, the distal tip 1340 of the polymeric tube 1310 has a profile that that corresponds geometrically with a profile of the distal ring tip 1380. When viewed from the side, it can be seen that the shape of the distal tip 1340 is the same as the shape of the distal ring tip 1380. Thus, the distances 1300 from the distal tip 1340 of the polymeric tube 1310 to the distal ring tip 1380 measured at different circumferential positions at the polymeric tube 1310 are the same.

FIGS. 15A-15B, show another example of the polymeric tip 270. The polymeric tube 1310 comprises a plurality of flaps (or flanges) 1400 disposed circumferentially around the longitudinal axis 1328 of the catheter 10 (or the axis 340 of the marker device 240). The flaps 1400 of the polymeric tube 1310 are configured to move radially away from the longitudinal axis 1328 of the catheter 10. The flaps 1400 are advantageous because they provide more surface area for contacting item(s) 1700 inside the patient (e.g., for grasping clot, tissue, etc.). Also, in some cases, the length 1500 (measured in a direction that is parallel to the longitudinal axis 1328) of the flap 1400 may be configured so that it is less than 50% of the cross-sectional dimension (e.g., diameter) of the lumen of the catheter 10. In such cases, the flaps 1400 are advantageous because even if they fold inward (collapse), they will not completely close the lumen of the catheter 10.

It should be noted that the flaps 1400 are not limited to having the shapes shown in the example, and that each flap 1400 may have a different shape from that illustrated. The polymeric tube of the polymeric tip 270 may include protrusions (e.g., flaps 1400) of any shape disposed circumferentially around the longitudinal axis of the catheter 10. For example, each protrusion may have a petal shape, a square shape, a partial circle shape, a partial elliptical shape, a rectangular shape, an elongated shape, etc. The protrusions are configured to move radially away from the longitudinal axis of the catheter 10 during use in order to grasp item(s) inside a patient.

In some cases, the protrusions (e.g., flaps 1400) are configured to easily deflect, thereby providing an atraumatic and easily trackable catheter tip, while being less likely to collapse inwards (thereby reducing aspiration force). Also, in some cases, the length 1500 of each protrusion may be configured to create shorter protrusion. Such protrusion may distend radially outward during clot ingestion, but may not present a risk of collapse due to the shorter length 1500 of the protrusion. By means of non-limiting examples, the length 1500 of each protrusion may be less than 40%, less than 30%, less than 20%, less than 10%, or less than 5% of a cross-sectional dimension of the polymeric tip 270.

FIG. 16 illustrates another example of the polymeric tip 270. In the illustrated embodiments, the distal tip 1340 of the polymeric tube 1310 has a profile that does not correspond geometrically with a profile of the distal ring tip 1380. As a result, the distances 1300 from the distal tip 1340 of the polymeric tube 1310 to the distal ring tip 1380 measured at different circumferential positions at the polymeric tube 1310 are different. This configuration may exhibit better trackability by having a circumferential section of the polymeric tip 270 that is easier to deflect (compared to another circumferential section of the polymeric tip 270), while having another circumferential section of the polymeric tip 270 for preventing tip collapse (e.g., complete closure of the catheter lumen). One or more circumferential sections of the polymeric tip 270 may also be configured to accommodate higher aspiration force and/or clot ingestion.

In one or more embodiments described herein, the polymeric tip 270 may be configured for one or more performance requirements, such as trackability, prevention of tip collapse or invagination, clot ingestion, or any combination of the foregoing. For example, for access catheters, the tip geometry may be configured for trackability. For aspiration catheters, the tip geometry may be configured for aspiration performance.

### Polymeric Layers

As discussed, in one or more embodiments described herein, the catheter 10 may include a first polymeric layer 260 disposed circumferentially around the marker device 240 and/or at least a part of the tubular structure 200. FIG. 17 shows an example of a cross-section of the distal segment of the catheter 10. As shown in the figure, the catheter 10 includes the first polymeric layer 260 disposed circumferentially around an exterior surface of the ring structure 300 of the marker device 240. The first polymeric layer 260 extends proximally so that it also surrounds at least a part of the tubular structure 200. In some cases, the polymeric tip 270 is integral with the first polymeric layer 260. The polymeric tip 270 may be attached to the first polymeric layer 260 or may be formed together with the first polymeric layer 260.

As shown in FIG. 7, the catheter 10 further includes a second polymeric layer 262 disposed at an inner surface of the marker device 240 and an inner surface of the tubular structure 200. In some cases, the polymeric tip 270 is integral with the second polymeric layer 262. The polymeric tip 270 may be attached to the second polymeric layer 262 or may be formed together with the second polymeric layer 262.

In some cases, the first polymeric layer 260, the polymeric tip 270, and the second polymeric layer 262 may be integral with each other. In one implementation, the first polymeric layer 260, the polymeric tip 270, and the second polymeric layer 262 may be formed together. The first polymeric layer 260 and the second polymeric layer 262 may be made from a same material, or from different respective materials. Also, the polymeric tip 270 may be made from a same material as the first polymeric layer 260 and/or the second polymeric layer 270. In some cases, a first material of the first polymeric layer 260 may extend to the space that is distal to the marker device 240 to form a first part of the polymeric tip 270, and a second material of the second polymeric layer 262 may extend to the space that is distal to the marker device 240 to form a second part of the polymeric tip 270. In such cases, the polymeric tip 270 is made from the first material of the first polymeric layer 260 and from the second material of the second polymeric layer 262. The first material and the second material may be the same material, or may be different from each other.

In the above examples, the catheter 10 is described as having the marker device 240. In other cases, the catheter 10 may not include the marker device 240. In such cases, the polymeric tube of the polymeric tip 270 may comprise a proximal tube end that abuts the distal end of the tubular structure 200.

In one or more embodiments, the first polymer layer 260 may comprise a first segment and a second segment proximal the first segment. The first segment may be made from NEUSoft (e.g., NEUSoft 62A). In some cases, the second segment 262 may be made from Pebax (e.g., Pebax 25D, Pebax 35D, or Pebax 45D). Also, in some cases, the first polymer layer 260 may include a third segment. The second segment may be made from a first Pebax, and the third segment may be made from a second Pebax that is different from the first Pebax. In further cases, the first polymer layer 260 may further comprise a fourth segment. The second segment may be made from a first Pebax, the third segment may be made from a second Pebax that is different from the first Pebax and the fourth segment may be made from a third Pebax that is different from the first Pebax and that is different from the second Pebax.

In one or more embodiments, the second polymer layer 262 may comprise a first segment and a second segment proximal the first segment. The first segment of the second polymer layer 262 may be made from NEUSoft (e.g., NEUSoft 62A). The second segment of the second polymer layer 262 may be made from PTFE.

In some cases, the first segment of the first polymer layer 260 and a first segment of the second polymer layer 262 may be made from a same material (e.g., NEUSoft). The first segment of the first polymer layer 260 and the first segment of the second polymer layer 262 may be closer to a distal end of the catheter 10 than to a proximal end of the catheter 10.

Also, in some cases, a second segment of the first polymer layer 260 and a second segment of the second polymer layer 262 are made from different respective materials, wherein the second segment of the first polymer layer 260 is proximal to the first segment of the first polymer layer 260, and wherein the second segment of the second polymer layer 262 is proximal to the first segment of the second polymer layer 262.

### Tubular Structure

It should be noted that the tubular structure 200 may be any member having a tube configuration. The tubular structure 200 is configured to provide axial stiffness, bending stiffness, torsional stiffness, or any combination of the foregoing, for the catheter 10. The tubular structure 200 may be a braid formed by a plurality of braid wires. Alternatively, the tubular structure 200 may be one or more coils. As a further alternative, the tubular structure 200 may be formed by removing some materials from a tube to create openings (e.g., elongated slots) through a wall of the tube. The created openings may be cutouts that provide flexibility for the catheter 10.

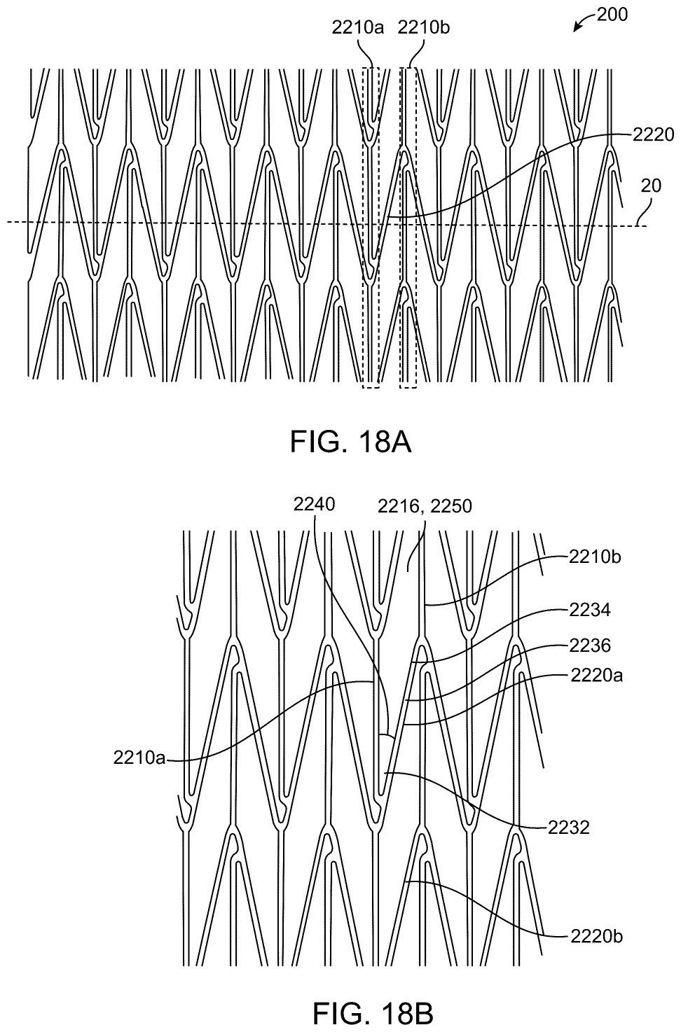

FIGS. 18A-18B illustrate a part of a tubular structure 200 of the catheter 10 of FIG. 1. The tubular structure 200 has a plurality of ring elements 2210 arranged in series along the longitudinal axis 20. In the illustrated embodiments, the ring elements 2210 are respective closed-loops. In FIGS. 18A-18B, two of the ring elements 2210 are identified (e.g., a first ring element 2210a and a second ring element 2210b). The ring elements 2210 lie within respective planes that are arranged in series along the longitudinal axis 20. The planes in which the ring elements 2210 lie are substantially perpendicular (e.g., 90 degrees+/−10 degrees) to the longitudinal axis 20, when the tubular structure 200 is in a relaxed state. In some embodiments, a ring element 2210 may be considered as lying within a plane if at least 20%, or at least 30%, or at least 40%, or at least 50%, or at least 60%, or at least 70%, or at least 80%, or at least 90% (e.g., 100%), of a circumferential length of a part of the ring element 2210 lies within a plane.

The tubular structure 200 also includes connecting members 2220 connected between adjacent ones of the ring elements 2210. As shown in FIG. 18B, the tubular structure 200 includes multiple connecting members 2220a, 2220b connected between adjacent ring elements 2210a, 2210b. The connecting member 2220a includes a first member end 2232, a second member end 2234 opposite from the first member end 2232, and a member body 2236 extending between the first member end 2232 and the second member end 2234. The member body 2236 forms an acute angle 2240 with respect to the ring element 2210a. In some cases, the angle 2240 may be measured with the tubular structure 200 being “un-rolled” to a flat configuration. Also, the first member end 2232 of the connecting member 2220a and the second member end 2234 of the connecting member 2220a define a line that is non-parallel to the longitudinal axis 20 of the tubular structure 200.

In the illustrated embodiments, the connecting member 2220 has a rectilinear configuration. In other embodiments, at least a part of the connecting member 2220 may have a curvilinear configuration.

In other embodiments, the tubular structure 200 may include more than two connecting members 2220 between adjacent ring elements 2210. In further embodiments, the tubular structure 200 may include only one connecting member 2220 between adjacent ring elements 2210.

In other embodiments, the ring elements 2210 and/or connecting member 2220 may have variations in length, width and thickness to adjust flexibility and kink resistance of the tubular structure 200.

In some embodiments, the member body 2236 of each connecting member 2220 rotates and/or bends relative to the adjacent ring element 2210 as the tubular structure 200 is being axially loaded (e.g., tensioned or compressed). Accordingly, the angle 2240 changes as the tubular structure 200 is being axially loaded. In addition, as the tubular structure 200 is being axially loaded to change (e.g., to increase or decrease) a spacing between adjacent ring elements 2210, the cross-sectional shape of the ring elements 2210 are maintained.

Also, in some embodiments, the member body 2236 of each connecting member 2220 rotates and/or bends relative to the adjacent ring element 2210 as the tubular structure 2200 is being bended. Accordingly, the angle 2240 changes as the tubular structure 200 is being bended. In addition, as the tubular structure 200 is being bended, the cross-sectional shape of the ring elements 2210 is maintained.

In some embodiments, filler 2250 may be disposed in the spacing 2216 defined between the ring elements 2210 and the connecting members 220. The filler 2250 provides a seal to prevent fluid from passing across a wall of the tubular structure 200.

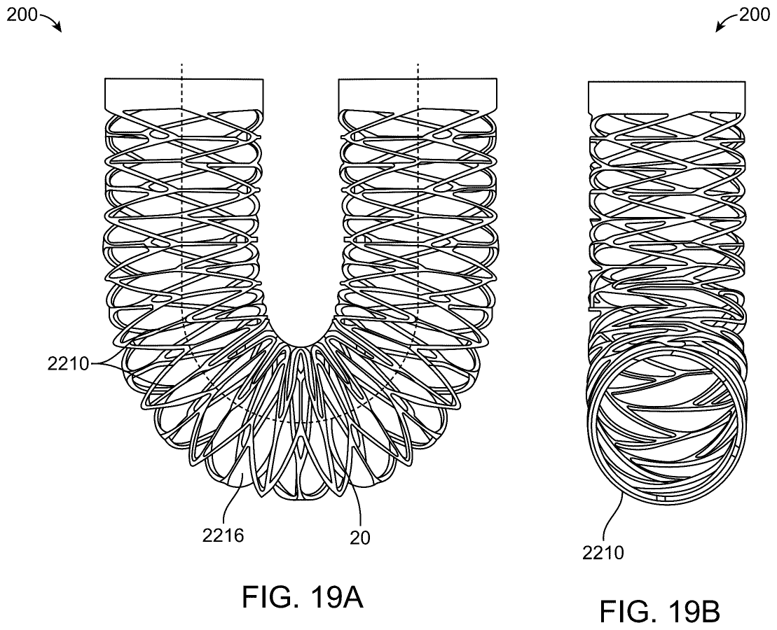

FIGS. 19A-19B illustrate a bending of the tubular structure 200 of FIG. 18A. As shown in FIG. 19A, when the tubular structure 200 is being bent, the ring elements 2210 stay substantially perpendicular (e.g., 90 degrees+/−10 degrees) to the longitudinal axis 20. The connecting members 2220 are configured to move relative to the ring elements 2210 in correspondence with the bending of the tubular structure 200. This allows the connecting members 2220 to conform to a change in a spacing distance between adjacent ring elements 2210 due to the bending of the tubular structure 200. As shown in FIG. 19B, the tubular structure 200 is advantageous because the close-loop ring elements 2210 prevents the tubular structure 200 from collapsing radially inward. Thus, the cross-sectional shape of the ring elements 2210 are maintained even during bending of the tubular structure 200, and kinking of the catheter 10 is prevented.

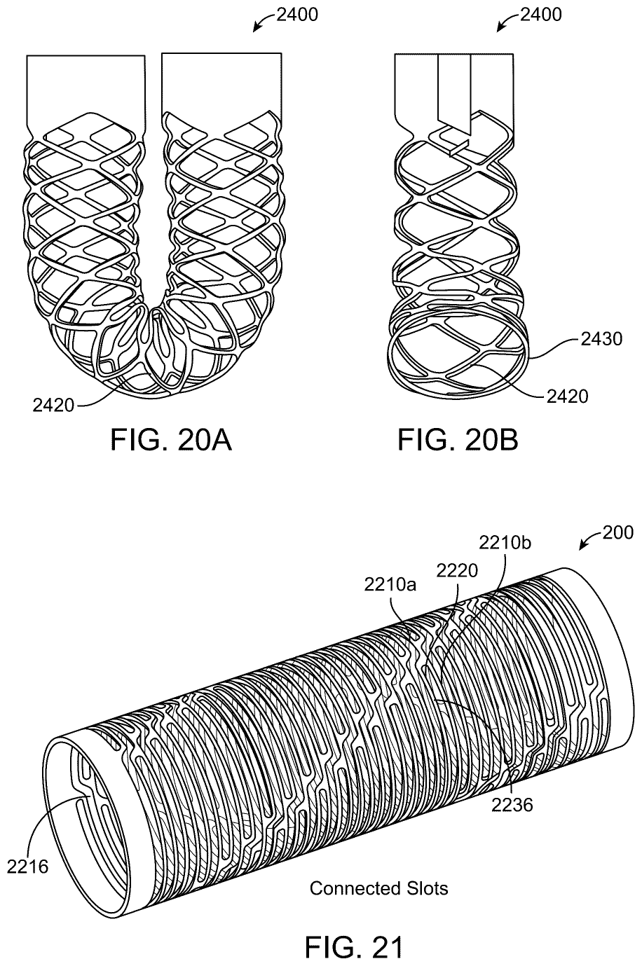

FIGS. 20A-20B illustrate a bending of another tubular structure 2400 that is different from the tubular structure 200 of FIGS. 18A-18B. The tubular structure 2400 comprises members 2420 that are arranged in crisscross configuration. Unlike the tubular structure 200, the tubular structure 2400 does not have any closed-loop ring elements that lie in respective planes. As shown in FIG. 20B, during bending of the tubular structure 2400, the tubular structure 2400 may be “squished” due to one side of the tubular structure 2400 being in compression from the bending. As a result, if the tubular structure 2400 defines a circular cross-section lumen while in a relaxed state, the “squished” tubular structure 2400 may result in the lumen having an elliptical shape 2430. A catheter constructed using the tubular structure 2400 will be easily kinked during use.

In other embodiments, instead of having the acute angle 2240 shown in the above example of FIGS. 18A-18B, the member bodies 2236 of the connecting members 2220 and their respective adjacent ring elements 2210 of the tubular structure 200 may form other non-zero acute angles when the tubular structure 200 is in a relaxed state. Also, in other embodiments, the member bodies 2236 (or at least a part of the member bodies 2236) of the connecting members 2220 may be parallel with their respective adjacent ring elements 2210.

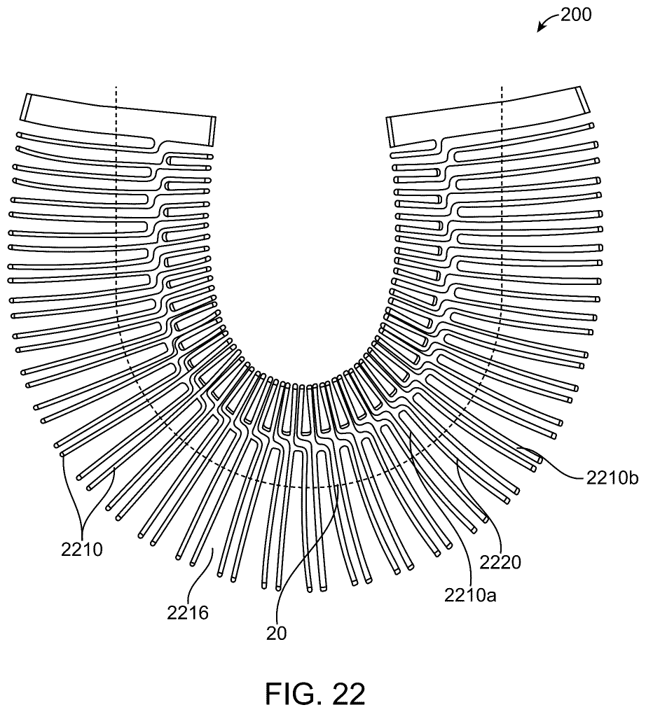

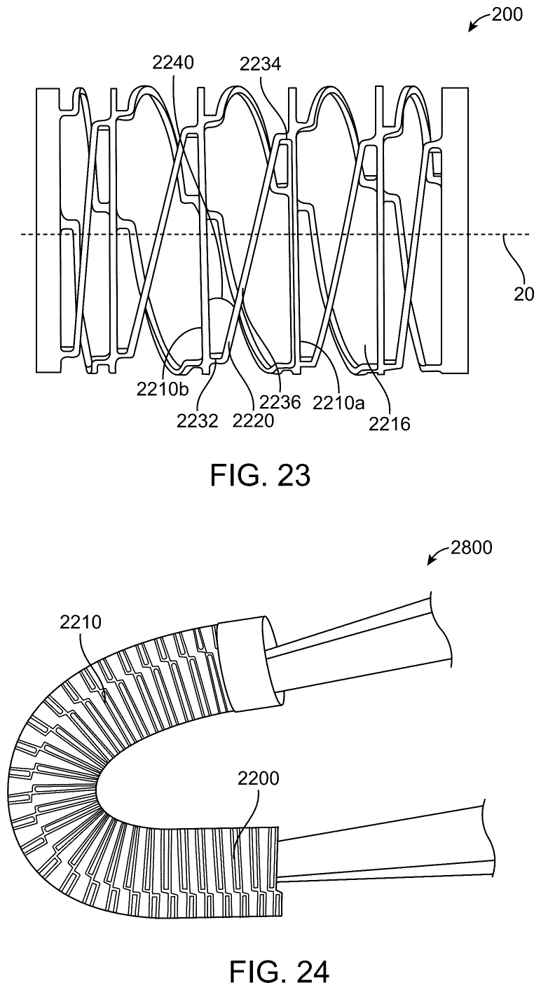

FIG. 21 illustrates another tubular structure 200. The tubular structure 200 is similar to that of FIGS. 18A-18B, except that a majority of the body 2236 of each connecting member 2220 is parallel to an adjacent ring element 2210 when the tubular structure 200 is in a relaxed state. In particular, a majority of each connecting member 2220 lies in a plane that is parallel to the plane in which an adjacent ring element 2210 lies when the tubular structure 200 is in a relaxed state

The tubular structure 200 of FIG. 21 may be considered as a variation of the tubular structure 200 of FIGS. 18A-18B.

FIG. 22 illustrates a bending of the tubular structure 200 of FIG. 21. As shown in FIG. 22, when the tubular structure 200 is being bent, the ring elements 2210 stay substantially perpendicular (e.g., 90 degrees+/−10 degrees) to the longitudinal axis 20. The connecting members 2220 are configured to move (e.g., flex and/or rotate) relative to the ring elements 2210 in correspondence with the bending of the tubular structure 200. This allows the connecting members 2220 to conform to a change in a spacing distance between adjacent ring elements 2210 due to the bending of the tubular structure 200. As similarly discussed, the tubular structure 200 is advantageous because the closed-loop ring elements 2210 prevent the tubular structure 200 from collapsing radially inward. Thus, the cross-sectional shape of the ring elements 2210 are maintained even during bending of the tubular structure 200, and kinking of the catheter 10 is prevented.

FIG. 23 illustrates a tensioning of the tubular structure 200 of FIG. 21. As the tubular structure 200 is being tensioned, the planes in which the respective ring elements 2210 (e.g., ring elements 2210a, 2210b) lie remain substantially perpendicular (e.g., 90 degrees+/−10 degrees) to the longitudinal axis 20. The connecting members 2220 between adjacent pairs of the ring elements 2210 elastically flex in correspondence with axial movement of the ring elements 2210 as the tubular structure 200 is being tensioned. As shown in the figure, the member body 2236 of each connecting member 2220 rotates and/or bends relative to the adjacent ring element 2210 so that the angle 2240 changes as the tubular structure 200 is being tensioned. In addition, as the tubular structure 200 is being tensioned to change a spacing between adjacent ring elements 2210, the cross-sectional shape of the ring elements 2210 are maintained.

FIG. 24 illustrates a tube segment 2800 having the tubular structure 200 of FIG. 21, particularly showing the tube segment 2800 being bent. The tube segment 2800 may be a part of the catheter 10 of FIG. 1 in some embodiments. As shown in FIG. 24, the tube segment 2800 with the tubular structure 200 can undergo very tight bending to have a bent shape with small radius of curvature. Through the entire length of the bent, the gaps between respective pairs of adjacent ring elements 2210 remain substantially even (e.g., the gaps between different pairs of adjacent ring elements 2210 do not vary by more than 10%). This is the case for both the gaps on the tension side, and the gaps on the compression side, of the bent tube segment 2800.

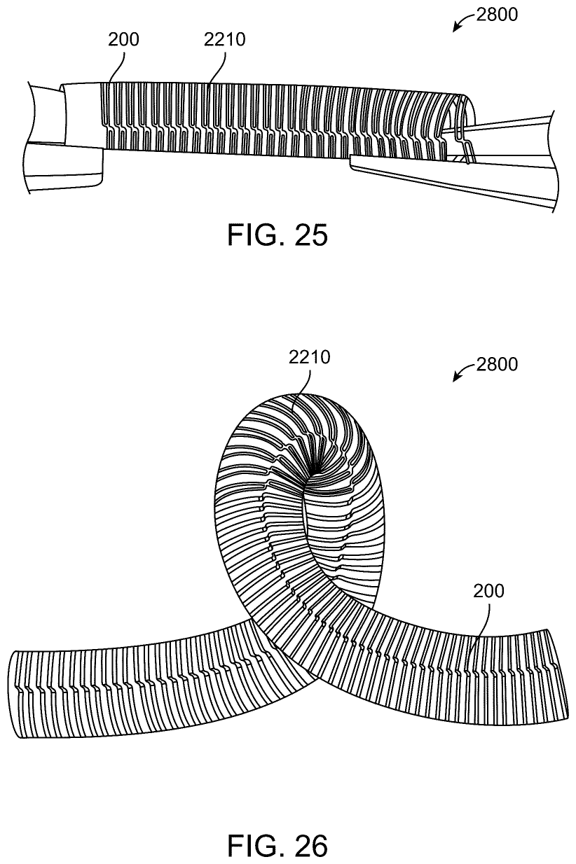

FIG. 25 illustrates the tube segment 2800 of FIG. 24, particularly showing the tube segment 2800 being tensioned. While the tube segment 2800 is being tensioned, the gaps between respective pairs of adjacent ring elements 2210 remain substantially even (e.g., the gaps between different pairs of adjacent ring elements 2210 do not vary by more than 10%).

FIG. 26 illustrates the tube segment 2800 of FIG. 24, particularly showing the tube segment 2800 being tested for kink resistance. As shown in FIG. 26, the tube segment 2800 with the tubular structure 200 can undergo 360-degree bending to have a bent shape with small radius of curvature. Through the entire length of the bent, the gaps between respective pairs of adjacent ring elements 2210 remain substantially even (e.g., the gaps between different pairs of adjacent ring elements 2210 do not vary by more than 10%). This is the case for both the gaps on the tension side, and the gaps on the compression side, of the bent tube segment 2800. As shown in the figure, even with such extreme bending, the tube segment 2800 forms no kink, and the structural integrity of the tube segment 2800 is maintained.

In any of the embodiments of the tubular structure 200 described herein, the tubular structure 200 may be made from a raw tube. The raw tube may be a metal tube, an alloy tube, a plastic tube, a polymeric tube, or a tube made from any of other materials. The raw tube is then cut to form the ring elements 2210 and connecting members 2220. In such cases, the ring elements 2210 and the connecting members 2220 are parts of a cut tube. The cutting of the raw tube may be performed using laser cutting in some embodiments. For example, an electronic file storing geometric information (e.g., shape information, dimension information, etc.) regard the tubular structure 200 to be formed may be created. The electronic file may be provided to a processing unit of a laser cutting machine. The processing unit processes the electronic file to operate a laser cutter of the laser cutting machine to cut geometric pattern(s) defined by the information in the electronic file. In some embodiments, the laser cutting may be performed on the raw tube. In other embodiments, instead of a raw tube, a raw sheet of material may be provided, and the laser cutting may be performed on the raw sheet. After the laser cutting is performed, the cut sheet may be rolled to form the tubular structure 200. Edges (that are parallel to the longitudinal axis) of the rolled sheet may be connected together to form a closed loop tube. Other techniques involve forming the desired pattern into a sheet or a tube by chemical etching or electrical discharge machining.

In other embodiments, the ring elements 2210 and the connecting members 2220 may be integrally formed together. For example, a mold having a rod with protrusions on the surface of the rod may be provided. The protrusions correspond with the space 216 to be formed between the ring elements 2210 and the connecting members 2220. Then the material for forming the ring elements 2210 and the connecting members 2220 is deposited onto the mold. The material is then cured to form the ring element 2210 and the connecting members 2220.

In further embodiments, the ring elements 2210 and the connecting members 2220 may be separately formed, and are then connected to each other after they are formed. In one implementation, a plurality of ring elements 2210 may be provided. Then a tubular mesh may be used to connect the ring elements 2210 in series. In particular, the ring elements 2210 in spaced-away configuration may be disposed over the tubular mesh, and are arranged in series along a longitudinal axis of the tubular mesh. The ring elements 2210 may then be secured to the tubular mesh, such as via an adhesive, glue, weld, etc. Parts of the tubular mesh between the ring elements 2210 become and function as the connecting members 2220.

As discussed, in some embodiments, the catheter 10 may include an outer layer 260 (e.g., an outer sheath) disposed circumferentially around the tubular structure 200, and/or an inner layer 262 (e.g., an inner sheath) disposed at an inner surface of the tubular structure 200. If the catheter 10 includes both the outer layer 260 and the inner layer 262, the tubular structure 200 is sandwiched there-between.

In some cases, material of the outer layer 260 and/or material of the inner layer 262 may fill the space 2216 defined by elements of the wall of the tubular structure 200. In such cases, the material from the outer layer 260 and/or the material from the inner layer 262 may form a filler (e.g., the filler 2250) that fills the space at the wall of the tubular structure 200.

For example, a filler may be disposed in the space (e.g., gaps) 216 between the ring elements 2210 and the connecting members 2220. In some embodiments, the tubular structure 200 may be dipped into a polymeric solution to allow the polymeric solution to fill the space 2216. The polymeric solution may then be cured to form the filler. Excess filler material may be removed using agent, by cutting, sanding, etc.

Also, in some embodiments, the filler 2250 occupying the space 2216 may have the same thickness as the thickness of the wall of the tubular structure 200. In other embodiments, the filler 2250 may be thicker than the thickness of the wall of the tubular structure 200.

In addition, in some embodiments, the material of the filler 2250 may extend pass the exterior surface of the tubular structure 200. In some cases, the material of the filler 2250 may be disposed on the exterior surface of the tubular structure 200 to form an outer layer (e.g., layer 260) covering the tubular structure 200. The outer layer may be formed integrally together with the filler in the space 2216. In other embodiments, the outer layer may be formed separately from the filler 2250, and is disposed on the exterior surface of the tubular structure 200 after the filler 2250 is disposed (e.g., formed) in the space 2216. In such cases, the outer layer may be made from the same material as the filler 2250, or may be made from a material that is different from the filler 2250.

Similarly, in some embodiments, the material of the filler 2250 may extend pass the inner surface of the tubular structure 200. In some cases, the material of the filler 2250 may be disposed on the inner surface of the tubular structure 200 to form an inner layer (e.g., 260) covering the inner wall of the tubular structure 200. The inner layer may be formed integrally together with the filler 2250 in the space 2216. In other embodiments, the inner layer may be formed separately from the filler 2250, and is disposed on the inner surface of the tubular structure 200 after the filler 2250 is disposed (e.g., formed) in the space 2216. In such cases, the inner layer may be made from the same material as the filler 2250, or may be made from a material that is different from the filler 2250.

In addition, in some embodiments, the ring elements 2210 may have the same thickness as the connecting members 2220. In other embodiments, the ring elements 2210 and the connecting members 2220 may have different thicknesses. For example, in other embodiments, the ring elements 2210 may have a first thickness, and the connecting members 2220 may have a second thickness, wherein the first thickness may be larger or smaller than the second thickness.

### Tube Manufacturing Process

Various techniques may be employed to make a tube having the tubular structure 200 for the catheter 10. In some embodiments, a coating material may be applied so that the material is disposed in the openings of the tubular structure 200 and forms an outer layer covering the exterior surface of the tubular structure 200. The coated tubular structure 200 forms the tube. In other embodiments, the coating material may encapsulate the tubular structure 200 to form the tube. In such cases, the coating material covers the exterior surface of the tubular structure, covers the interior surface of the tubular structure, as well as filling the openings (e.g., slots) through the wall of the tubular structure 200.

In some embodiments, the coating material forming part of the tube may have a modulus of elasticity that is lower than the modulus of elasticity of the tubular structure 200. For example, the coating material may have a modulus of elasticity that is less than 50%, or more preferably less than 30%, or more preferably less than 20%, or more preferably less than 10%, or more preferably less than 5%, or more preferably less than 1%, of that of the tubular structure 200. In one implementation, the coating material may have a modulus of elasticity that is less than 15 Mpa (e.g., 10 Mpa or less).

Also, the coating material forming part of the tube may have the ability to undergo significant elongation before break point. For example, in some embodiments, the coating material may be capable of having a strain (defined as an amount of elongation of the material divided by the length of the material) of at least: 20%, 40%, 60%, 80%, 100%, 200%, 300%, 400%, 500%, 600%, 700%, 800%, 900%, 1000%, or greater.

Various materials may be utilized as the coating material. By means of non-limiting examples, the coating material may include polyurethane, polyurethane-based material, silicon-based material, any material having polyurethane dispersion or silicon-based dispersion, etc. Examples of coating material that may be used include CD102® or AD111® from Covestro, Gelest Ex-sil50® from Gelest, NEUSoft (e.g., NEUSoft 62A), Pebax (e.g., Pebax 25D, Pebax 35D, or Pebax 45D), etc.

In some embodiments, because the coating material is significantly softer than the material of the tubular structure 200, the resulting tube will have one or more mechanical properties that are contributed predominantly (e.g., more than 50%, more than 60%, more than 70%, more than 80%, more than 90%, more than 95%, more than 99%, etc.) by the tubular structure 200. By means of non-limiting examples, the one or more mechanical properties may be a bending stiffness, an axial stiffness, a torsional stiffness, a shear stiffness, or any combination of the foregoing.

In one or more embodiments, the tube may optionally further include a hydrophilic coating that is disposed on the exterior surface of the tube, and/or on the interior surface of the tube. In some cases, an initial coating material may first be applied on the tubular structure 200 to fill the openings at the wall of the tubular structure 200, and optionally to cover the exterior and/or interior surface of the tubular structure 200. Then the hydrophilic coating is applied over the initial coating material.

Constructing the tube using the tubular structure 200 (providing a majority of the mechanical properties), and using the very soft coating material, is advantageous because it prevents the distal end of the tube from being too stiff, and because it allows design of catheter to be easier with resulting behavior of the catheter being more predictable (because computational modeling of the catheter may be made based on the tubular structure design only).

Various techniques may be employed to apply the coating material to the tubular structure 200 in different embodiments. For example, in some embodiments, a deposition technique may be employed to deposit the coating material onto the tubular structure 200. In other embodiments, a dipping process may be utilized to apply the coating material onto the tubular structure 200.

In some embodiments, when using a dipping technique to apply the coating material onto the tubular structure 200, a barrier may be provided inside the central lumen of the tubular structure 200 to prevent the coating material from entering into the central lumen. For example, in some embodiments, the interior surface of the tubular structure 200 may be masked to prevent the coating material from outside the tubular structure 200 from flowing into the central lumen through the openings at the wall of the tubular structure 200. In other embodiments, a tube or rod may be placed inside the central lumen of the tubular structure 200 to act a barrier for preventing the coating material from entering into the central lumen. The tube or the rod may be made from Ptfe, HDPE, stainless steel, or any of other suitable materials.

The tubular structure 200 may then be placed into a reservoir of the coating material in liquid or viscous form. The coating material may be tailored to have certain specific viscosity to assist with the coating process. In some cases, the tubular structure 200 may be placed vertically into the reservoir with the longitudinal axis of the tubular structure 200 forming an angle that is 90°+/−25° with respect to a top surface of the liquid in the reservoir. In other cases, the tubular structure 200 may be placed horizontally into the reservoir with the longitudinal axis of the tubular structure 200 forming an angle that is 0°+/−25° with respect to a top surface of the liquid. In other embodiments, the tubular structure 200 may be placed into the reservoir at other angles that are different from the above examples.

In some embodiments, the tubular structure 200 may be inserted into the reservoir at a certain rate, such as anywhere from 0.01 cm/sec to 5 cm/sec. In other embodiments, the tubular structure 200 may be inserted at a rate that is slower or faster than the above range of rates. Also, in some embodiments, as the tubular structure 200 is inserted into the reservoir, the tubular structure 200 may be rotated at a certain rate, such as 2-10 rotations per minute. The rotational speed may be slower or faster than 2-10 rotations per minute in other embodiments. In further embodiments, the tubular structure 200 may not be rotated as it is being inserted into the reservoir.

After the tubular structure 200 is dipped into the reservoir of the coating material, the tubular structure 200 may then be removed from the reservoir. In some embodiments, the tubular structure 200 may be removed from the reservoir at a certain rate, such as anywhere from 0.01 cm/sec to 10 cm/sec. In other embodiments, the tubular structure 200 may be removed from the reservoir at a rate that is slower or faster than the above range of rates. Also, in some embodiments, as the tubular structure 200 is removed from the reservoir, the tubular structure 200 may be rotated at a certain rate, such as 2-10 rotations per minute. The rotational speed may be slower or faster than 2-10 rotations per minute in other embodiments. In further embodiments, the tubular structure 200 may not be rotated as it is being removed from the reservoir of the coating material.

As a result of dipping the tubular structure 200 into the reservoir and removing it from the reservoir, a first layer (e.g., layer 260) of coating material is disposed on the exterior surface of the tubular structure 200. The coating material also spans and fills up the openings at the wall of the tubular structure 200.

After the tubular structure 200 is removed from the reservoir, the tubular structure 200 with the coating material is held at certain time and at certain temperature to solidify the coating material. The time for the coating material to solidify may be anywhere from 1 minute to 120 minutes. In other embodiments, the solidifying time may be faster than 1 minute, or longer than 120 minutes. Also, in some embodiments, the temperature for solidifying the coating material may be anywhere from 20° C. to 100° C. In other embodiments, the temperature for solidifying the coating material may be less than 20° C. or higher than 100° C.

In some embodiments, the insertion of the tubular structure 200 into the reservoir, and the removing of the tubular structure 200 from the reservoir, may be repeated one or more times (e.g., anywhere from 1 additional time to 30 additional times, or more) until a desired thickness for the coating material is achieved. In some embodiments, the thickness of the coating material created on the exterior surface of the tubular structure 200 may be anywhere from 0.0001 inch to 0.003 inch, or greater.

In some embodiments, the applying of the coating material onto the tubular structure 200 may be performed in a vacuum. For example, in some embodiments, the reservoir of coating material may be placed in a vacuum chamber, and the dipping and removing of the tubular structure 200 may be performed inside the vacuum chamber. The solidifying of the coating material may also occur inside the vacuum chamber.

In some embodiments, after the coating material is solidified, a hydrophilic coating may be applied on the solidified coating. The application of the hydrophilic coating may be performed using a dipping technique that is similar to that described above. In other embodiments, the hydrophilic coating may be applied on the solidified coating using a deposition technique. The hydrophilic coating may be applied to the exterior surface of the tube 11 and/or to the interior surface of the tube 11. In some cases, the hydrophilic coating may be considered to be a part of the tube 11.

It should be noted that the processing of coating the tubular structure 200 is not limited to the examples described above, and that the tubular structure 200 may be coated using other techniques, or variations of the techniques described. For example, in other embodiments, a barrier may not be provided inside the central lumen of the tubular structure to prevent the coating material from entering into the central lumen. Instead, coating material is allowed to flow into the central lumen during the dipping process. In such case, after the tubular structure 200 is removed from the reservoir, and before the coating material in the central lumen of the tubular structure 200 solidifies, a plunger may be placed inside the central lumen and be moved longitudinally through the tubular structure 200 to remove excess material inside the central lumen. In some embodiments, all coating material inside the central lumen is removed so that there is no coating material disposed on the interior surface of the tubular structure 200. In other embodiments, some but not all of the coating material inside the central lumen is removed so that a layer (e.g., layer 262) of the coating material remains on the interior surface of the tubular structure 200. In further embodiments, the removing of the excess coating material inside the central lumen of the tubular structure 200 may be removed using a cutter after the coating material has been solidified.

In other embodiments, before the tubular structure 200 is inserted into the reservoir of coating material, a rod or a tube (smaller than a size of the central lumen of the tubular structure 200) may be placed inside the central lumen of the tubular structure 200. The rod or the tube has an exterior surface that is spaced away from the inner surface of the tubular structure 200. This allows the coating material to fill the space between the rod/tube and the interior surface of the tubular structure 200, thereby creating a layer (e.g., layer 262) of coating on the interior surface of the tubular structure 200. The coating material also fills the openings at the wall of the tubular structure 200, and extends to outside the tubular structure 200 to create a layer (e.g., layer 260) of coating on the exterior surface of the tubular structure 200.

In further embodiments, instead of using the dipping technique, the coating material may be pumped to encapsulate the tubular structure 200 at certain flow rates to create a desired thickness of the coating.

As used in this specification, the term “relaxed state” (e.g., a relaxed state of the tubular structure, a relaxed state of the catheter, etc.) refers to a state of an object in which no external force is applied against the object (other than the force due to gravity). For example, a relaxed state of a tubular structure/a catheter may refer to a state of the tubular structure/catheter that is placed on a surface without having bending force, axial force, and torsional force applied to the tubular structure/catheter.

Although particular embodiments have been shown and described, it will be understood that it is not intended to limit the claimed inventions to the preferred embodiments, and it will be obvious to those skilled in the art that various changes and modifications (e.g., the dimensions and/or shapes of various parts) may be made without department from the spirit and scope of the claimed inventions. The specification and drawings are, accordingly, to be regarded in an illustrative rather than restrictive sense. The claimed inventions are intended to cover alternatives, modifications, and equivalents.

1. A marker device, comprising:
a ring structure having a distal end, a proximal end, and a body extending between the distal end and the proximal end, wherein the ring structure is made from a radiopaque material;
wherein the distal end of the ring structure comprises protruding elements disposed circumferentially around an axis of the ring structure; and
wherein the proximal end of the ring structure is configured to couple with, or extends from, a tubular structure, wherein the proximal end of the ring structure is distal to the tubular structure;
wherein each of the protruding elements has a length and a width, wherein the length is measured parallel to a longitudinal axis of the marker device and is shorter than the width, and
wherein the distal end of the ring structure has a sinusoidal profile extending circumferentially around the longitudinal axis of the marker device.

2. The marker device of claim 1, wherein the proximal end of the ring structure comprises tabs disposed circumferentially around the longitudinal axis of the marker device, and wherein the tabs are configured to be welded to, or extend from the tubular structure.

3. The marker device of claim 1, wherein the protruding elements comprise respective curvilinear tip surfaces.

4. The marker device of claim 3, wherein the distal end of the ring structure further comprises curvilinear trough surfaces, and wherein each of the curvilinear trough surfaces is disposed between two adjacent ones of the curvilinear tip surfaces.

5. The marker device of claim 4, wherein the curvilinear tip surfaces and the curvilinear trough surfaces together form a sinusoidal profile extending circumferentially around the longitudinal axis of the marker device.

6. The marker device of claim 1, wherein a ratio calculated by dividing the length by a total longitudinal length of the ring structure is at least 0.5.

7. The marker device of claim 1, wherein the ring structure has a plurality of holes disposed circumferentially at the body of the ring structure.

8. The marker device of claim 7, wherein at least one of the holes has an elongated configuration extending at least partially around the longitudinal axis of the marker device.

9. A catheter comprising the marker device of claim 7, and a first polymer layer disposed over an exterior surface of the ring structure, wherein parts of the first polymer layer extend into the holes of the ring structure.

10. The catheter of claim 9, wherein the first polymer layer extends distally past a distal tip of the marker device to form a polymeric tip.

11. The catheter of claim 9, further comprising a second polymer layer disposed at an interior surface of the ring structure.

12. A catheter comprising the marker device of claim 1, and the tubular structure, wherein the tubular structure is configured to provide axial stiffness, bending stiffness, torsional stiffness, or any combination of the foregoing, for the catheter.

13. The catheter of claim 12, further comprising a first polymer layer disposed over an exterior surface of the ring structure, and over at least a part of an exterior surface of the tubular structure, wherein the first polymer layer extends distally past a distal tip of the marker device to form a polymeric tip.

14. The catheter of claim 13, wherein the polymeric tip comprises a polymeric tube that is distal to the marker device.

15. The catheter of claim 14, wherein the polymeric tube comprises a distal tip extending circumferentially around a longitudinal axis of the catheter.

16. The catheter of claim 15, wherein the distal tip of the polymeric tube has a profile that corresponds geometrically with a profile of the distal tip of the marker device.

17. The catheter of claim 15, wherein the distal tip of the polymeric tube has a profile that does not correspond geometrically with a profile of the distal tip of the marker device.

18. The catheter of claim 14, wherein the polymeric tube comprises a plurality of flanges disposed circumferentially around a longitudinal axis of the catheter.

19. The catheter of claim 18, wherein the flanges of the polymeric tube are configured to move radially away from the longitudinal axis of the catheter.

20. The catheter of claim 15, wherein distances from the distal tip of the polymeric tube to the distal tip of the marker device measured at at least three different circumferential positions at the polymeric tube are different.
